(19) United States
(12) Patent Application Publication
(10) Pub. No.: US 2024/0099295 A1
(43) Pub. Date: Mar. 28, 2024

(54) CELL TREATMENT AGENT

(71) Applicant: AYA Inc., Shiga (JP)

(72) Inventors: Akeo HAGIWARA, Shiga (JP); Yosaku HAGIWARA, Osaka (JP)

(21) Appl. No.: 18/275,514

(22) PCT Filed: Feb. 9, 2022
(86) PCT No.: PCT/JP2022/005134

§ 371 Date: Aug. 2, 2023

(30) Foreign Application Priority Data
Feb. 9, 2021 (JP) 2021-019395
May 31, 2021 (JP) 2021-091443

(51) Int. Cl. A01N 1/02; C08B 37/00

(52) U.S. Cl. CPC A01N 1/02; C08B 37/0084

(57) ABSTRACT

A cell treatment agent contains at least one selected from the group consisting of alginic acid, heparins, dextran sulfate, and a proteolytic enzyme inhibitor, as an effective component.

### TECHNICAL FIELD

The present invention relates to a cell treatment agent, in particular, a cell treatment agent that is used in performing treatments of detaching and suspending cells, making cells dormant, maintaining viability, preventing death, and activating suspended cells, for cells in tissue or cultured cells of a living organ.

### BACKGROUND ART

In survival and proliferation of adherent cells that are the majority of cells (hereinafter simply referred to as “cells”) except for special cells (non-adherent cells) such as unicellular organisms, blood cells, and cancer cells, the cells survive, exert their functions, and proliferate while they adhere to a scaffold of an extracellular matrix constituting a body in the body, or a scaffold such as a wall of a culture vessel or a predetermined carrier in a culture substrate. When such cells are used for research or medical care, it is necessary to detach the cells from the scaffold and to suspend the cells without weakening the cells.

In operations of detaching and suspending the cells, it is common to utilize the cell detaching effect of a surfactant or a cell detachment enzyme such as trypsin. For example, for collection of only cells by detaching and suspending the cells from a living organ, there has been known a treatment method that includes digesting a component that makes the cells adhere to the scaffold with a cell detachment agent such as a cell detachment enzyme or a surfactant to detach and suspend the cells, and collecting the cells by washing out the cells from the living organ (see non-patent literature (NPL) 1 for a surfactant method using a surfactant). For detaching cells in culture from a predetermined scaffold, and suspending the cells, there has been known a treatment method of adding a liquid containing a cell detachment agent such as trypsin to the culture vessel to cause the cell detachment agent to act on the cultured cells, thus detaching and suspending the cells (NPL 2).

However, surfactants and cell detachment enzymes used in these detaching and suspending treatments also digest essential components of the cells themselves. Therefore, if the surfactant or the cell detachment enzyme continuously acts on the cells even after detachment and suspension, the suspended cells will weaken and eventually die. Thus, the surfactant or the cell detachment enzyme has cytotoxicity. In order to protect suspended cells from the cytotoxicity, the suspended cells are separated from the surfactant or the cell detachment enzyme after suspending the cells, or the separated cells are washed or the surfactant or the enzyme is inactivated to remove the cytotoxic effect of the surfactant or the cell detachment enzyme. However, such a treatment using a cell detachment agent having cytotoxicity is inevitably accompanied by cell death to a certain extent in the course of suspension. Also, in order to use suspended cells for research or medical care, it is required to maintain viability without weakening the cells during detachment and suspension.

In light of such a current situation, there has been developed a method of using a special polymer as a temperature-sensitive cell culture scaffold and heating a part of the polymer to a certain temperature to detach and suspend cells (NPL 3). However, high price due to use of a special polymer, need of a special temperature control device, difficulty in temperature control or the like and unstable result, unusability in a stereoscopic and complicated device for culturing a large amount of cells, impossibility to detach and suspend cells in tissue from a living organ, and the like have been pointed out for this method.

When cells are used for research or medical care, it is necessary to store and transport the cells safely. In such a case, cells are generally transported and stored while they are contained in a liquid for preservation and transportation (preservation and transportation liquid). Since the cells weaken hourly at room temperature (normal temperature), they are stored and transported in a refrigerated or frozen state, and returned to normal temperature before use.

Even in the refrigerated state or in the course of returning to normal temperature, the viability of the cells decreases and the cells die with time. For the purpose of cell protection to prevent cell death and maintain cell viability, a method of adding and mixing 10% fetal bovine serum (FBS) or autologous serum to the liquid for preserving and transporting cells has been conventionally performed. It is known that the viability of the cells is dramatically increased (cell protective effect) by mixing fetal bovine serum (FBS) or autologous serum, compared to the case where FBS or autologous serum is not mixed (NPL 4 to 6).

However, FBS or autologous serum is biologics. There are many problems, such as infection, allergies, operability, and ethics for addition of FBS or autologous serum. On the other hand, substances that adequately replace FBS and autologous serum are not currently known.

Meanwhile, cells, especially cells that are currently in an undifferentiated or low differentiated state and have high potentiality to proliferate, and regenerate and form a tissue or organ in the future, can occasionally unnecessarily differentiate during preservation, and can change to exhibit properties different from those of the tissue or organ that is intended to be regenerated and formed using the cells. Maintaining the undifferentiated or low differentiated state by making cell functions dormant and preventing the unnecessary differentiation over the period of preservation of the cells is an important matter in practice of regenerative medicine, but the means have not yet been established, and establishment of a solution is desired.

### CITATION LIST

### Non Patent Literature

[NPL 1] Crapo P M., et al., An overview of tissue and whole organ decellularization processes. Biomaterials 32, 3233-3243 (2011)
[NPL 2] Hayflick L., et al., Subculturing human diploid fibroblast cultures. Edited by Kruse, P. F., et al., Tissue Culture Methods and Applications. 220-223 Academic Press (New York) (1973)
[NPL 3] Yamato M., et al., Thermo-responsive culture dishes allow the intact harvest of multilayered keratinocyte sheets without dispase by reducing temperature. Tissue Engineering, 7, 473-480 (2001)
[NPL 4] Juan Jose' Dorantes-Aranda et al. Novel application of a fish gill cell line assay to assess ichthyotoxicity of harmful marine microalgae. Harmful Algae., 10, 366-373 (2011)
[NPL 5] Liang C., et al. Serotonin promotes the proliferation of serum3 deprived hepatocellular carcinoma cells via upregulation of FOXO3a. Molecular Cancer 12 (2013) (open access)
[NPL 6] Julia C. et al. Towards a xeno-free and fully chemically defined cryopreservation medium for maintaining viability, recovery, and antigen-specific functionality of PBMC during long-term storage. J. Immunol. Methods 382, 24-31 (2012)

### SUMMARY OF THE INVENTION

### Problems to be Solved by the Invention

As described above, in performing a treatment of detaching and suspending cells in tissue or cultured cells from a living organ or from a culture substrate of live cells, it is required to (a) prevent death of the cells, and to (b) obtain suspended cells in a state that viability of the cells is maintained, or, in other words, not to reduce the viability of the cells in detaching and suspending the cells.

Also, in preservation and transportation of cells, it is necessary to protect the cells to prevent death of the cells without reducing the viability of the cells being preserved and transported, in order to let the cells being preserved and transported satisfactorily exert their functions.

Also, it is necessary to maintain cells, especially cells that are currently in an undifferentiated or low differentiated state and are rich in the ability to proliferate and regenerate and form a tissue or organ in the future, in the undifferentiated or low differentiated state by making cell functions dormant and preventing the unnecessary differentiation over the period of preservation of cells.

Also, when suspended cells in a dormant state are adhered to a living organ, a culture substrate, or the like, it is desired that the cells can be easily adhered, and it is necessary to activate and proliferate the adhered cells.

However, in order for the detached and suspended cells to adhere to a scaffold that is a culture substrate such as a wall of a culture vessel or a predetermined carrier, for example, or in order to “plant” such cells, it is necessary to maintain the state of the cells in standing still with being in contact with the scaffold for 12 to 24 hours in an apparatus in which normal culture conditions are kept. If the contact is poor, the cells cannot be planted on the scaffold. In particular, in order to plant cells in specific local sites of a living body, it is necessary to keep the cells in contact with the scaffold of the living body for 12 to 24 hours.

For this purpose, typically, a scaffold such as a nonwoven fabric is brought into contact with cells for 12 to 24 hours outside the living body to adhere the cells to the nonwoven scaffold, and the cells together with the scaffold to which the cells adhere are planted in a desired location in the living body. However, such planting disadvantageously requires time and labor.

In light of the above, it is an object of the present invention to provide a technique enabling safe and easy performance of, for example, a detaching and suspending treatment, a treatment of making cells dormant (dormancy increases the protective effect, and also prevents unnecessary differentiation of cells and maintains an undifferentiated or low differentiated state), a protection treatment for preservation and transportation, and the like while preventing death of cells and maintaining viability of cells in tissue or cultured cells, and a technique enabling safe and easy performance of a cell activating treatment for planting cells in a dormant state (for example, suspended cells).

### Solution to the Problems

The present inventor diligently studied to solve the above-described problems, and found that the aforementioned problems can be solved by using at least one selected from the group consisting of alginic acid, heparins, dextran sulfate, and a proteolytic enzyme inhibitor. The present invention is summarized as follows.
(1) A cell treatment agent containing at least one selected from the group consisting of alginic acid, heparins, dextran sulfate, and a proteolytic enzyme inhibitor as an active ingredient.
(2) The cell treatment agent according to the above (1) containing at least one selected from the group consisting of a cell culture solution, an extracellular fluid replacement solution, and a maintenance infusion.
(3) The cell treatment agent according to the above (1) or (2), for cell dormancy of making a cell in tissue or a cultured cell dormant.
(4) The cell treatment agent according to the above (1) or (2), for cell protection of maintaining viability of a cell in tissue or a cultured cell to prevent death of the cell.
(5) The cell treatment agent according to the above (1) or (2), for cell preservation for maintaining a cell in an undifferentiated state or a low differentiated state.
(6) The cell treatment agent according to the above (1) or (2), for cell detachment and suspension of detaching and suspending a cultured cell from a scaffold of a living tissue or from a scaffold of a culture substrate, or detaching and suspending a cell in tissue of a living tissue from a scaffold of the living tissue.
(7) The cell treatment agent according to any of the above (1) to (5), the cell treatment agent being a liquid for cell preservation, tissue preservation, or organ preservation.
(8) A set reagent for awakening of a dormant cell, the set reagent being a combination reagent of the cell treatment agent according to any of the above (1) to (7) and a solid or liquid preparation containing a polyvalent cation.

The term “scaffold” means what is known as one of the three elements of regenerative medicine in the present technical field.

Adherent cells, which comprise a majority of cells that make up a living body, need to be in a state that the cells are adhered to a fixed base in order to perform their intrinsic functions (including proliferation). This base is called “scaffold” in regenerative medicine. Adherent cells cannot perform their intrinsic functions in the state that they are suspended in liquid. The scaffold can be an artificial scaffold or a scaffold in a natural state. Examples of the artificial scaffold include a wall of a laboratory dish and the like in an artificial substrate for cell culture such as a laboratory dish. Also, fibers that carry cells correspond to the artificial scaffold in the case of planting external cells into a body in a form that the cells are carried and adhered to artificial fibers or the like.

A body tissue or an organ of a living body is composed of cells and an extracellular matrix (including collagen fibers, proteoglycans, and the like) that surrounds the cells. Adherent cells exist adhering to the extracellular matrix and exert their functions in the living body. Examples of the scaffold in its natural state include extracellular matrix to which cells adhere in a living tissue.

Here, the three elements of regenerative medicine refer to “cells” that constitute a tissue or organ, “bioactive substances” that are signal factors for the function of cells, and “scaffolds” for enabling cells and bioactive substances to move.

“Dormancy” means that cells stop proliferation, respiration, metabolism, and differentiation state maintenance while having viability (stopping differentiation state maintenance also means maintaining a dedifferentiation state, namely maintaining a low differentiated or undifferentiated state), or means that cells stop the adhesion.

“Protection” for cells means maintaining viability, namely, preventing the loss of the ability to perform cellular functions and preventing death of cells.

Activation of suspended cells means starting the proliferation, respiration, metabolism, and differentiation state maintenance of the suspended cells in the above-described “dormant” state or in the above-described “protected” state, or restoring the adhesion, respiration, metabolism, and differentiation state maintenance of the suspended cells in the above-described “dormant” state or in the above-described “protected” state. Activation of cells means restarting the proliferation, respiration, metabolism, and differentiation state maintenance of the cells in the above-described “dormant” state or in the above-described “protected” state, or restoring the adhesion, respiration, metabolism, and differentiation state maintenance of the cells in the above-described “dormant” state or in the above-described “protected” state for dormant cells including unsuspended cells. The activation of cells is synonymous to the later-described “awakening”.

The viability means that the cell retains the ability to perform its intrinsic functions (including proliferation function, respiration, metabolism, and differentiation state maintenance) in the living body, in addition to a fact that the cell is alive without dying.

### Advantageous Effects of the Invention

According to the present invention, it is possible to provide a technique enabling safe and easy performance of, for example, a detaching and suspending treatment, a treatment of making cells dormant, a protection treatment for preservation and transportation, and the like while preventing death of cells and maintaining viability of cells in tissue or cultured cells, and a technique enabling safe and easy performance of a cell activating treatment for planting cells in a dormant state.

### BRIEF DESCRIPTION OF THE DRAWINGS

FIG. 1 shows a captured image of the surface of a potassium phosphate film when cells having been detached and suspended with dextran sulfate are cultured on a culture dish with the potassium phosphate film in Experimental Example 5.

FIG. 2 shows a captured image of the surface of a potassium phosphate film when cells having been detached and suspended with heparin sodium are cultured on a culture dish with the potassium phosphate film in Experimental Example 5.

FIG. 3 shows a captured image of the bottom surface of a culture dish when cells having been detached and suspended with dextran sulfate are cultured on a culture dish without a potassium phosphate film in Experimental Example 5.

FIG. 4 shows a captured image of the bottom surface of a culture dish when cells having been detached and suspended with heparin sodium are cultured on a culture dish without a potassium phosphate film in Experimental Example 5.

### DESCRIPTION OF EMBODIMENTS

A cell treatment agent according to an embodiment of the present invention contains at least one selected from the group consisting of alginic acid, heparins, dextran sulfate and a proteolytic enzyme inhibitor, as an active ingredient (hereinafter, also referred to as “active ingredient of treatment agent” or “active ingredient”).

Alginic acid is a linear polysaccharide composed of two types of monosaccharides, i.e., β-D-mannuronic acid and α-L-guluronic acid, which are found in various brown algae such as kelp and wakame throughout the world. The structure has a portion configured by an M block made up of 1,4 bonded β-(1-4)-D-mannuronic acid, a G block made up of 1,4 bonded α-(1-4)-L-guluronic acid, and an MG block in which mannuronic acid and guluronic acid are alternately 1,4 bonded. It is also known that alginic acid becomes a smooth sticky aqueous solution (colloidal solution) when being dissolved in water, the viscous property (viscosity) of this aqueous solution is proportional to the polymerization degree of the alginic acid, and the viscosity of the aqueous solution increases as the polymerization degree increases. Various types of such alginic acids having different viscosities are commercially available, and various types including those forming a general sticky aqueous solution, and those having a viscosity adjusted to be lower than that of a general one, can be used, and effective application differs depending on the viscosity. Hereinafter, description is made while taking the cases of the following three types (a) to (c) conveniently classified by viscosity of alginic acid as examples. (a) High viscosity type alginic acid: for example, alginic acid having a relatively high viscosity of 60 mPa·s or more at 20° C. in 1 wt % aqueous solution, (b) low viscosity type alginic acid: for example, alginic acid having a relatively low viscosity of 5 mPa·s or more and less than 60 mPa·s at 20° C. in 1 wt % aqueous solution, and (c) very low viscosity type alginic acid: for example, alginic acid having a very low viscosity of 5 mPa·s or less at 20° C. in 1 wt % aqueous solution, or 30 mPa·s or less at 20° C. in 10 wt % aqueous solution. (a) High viscosity type alginic acid having a high viscosity tends to exert its function more effectively than (b) low viscosity type and (c) very low viscosity type alginic acids in a solution prepared to be 5 mg/ml or less, for example, when the preservation time is relatively short of about 24 hours at a level of a room temperature (22° C.). (b) Low viscosity type alginic acid having a relatively low viscosity tends to exert its function more effectively than (a) high viscosity type and (c) very low viscosity type alginic acids in a solution prepared to be 0.5 to 10 mg/ml, for example, when the preservation time is relatively long of about 120 hours at a level of a room temperature (22° C.). (c) Very low viscosity type alginic acid having a very low viscosity tends to exert its function more effectively than (a) high viscosity type alginic acid and (b) low viscosity type alginic acid in a solution prepared to be 10 mg/ml or more, for example, when the preservation time under refrigeration (4° C.) is relatively long of about 168 hours. While various effects of alginic acid as a cell treatment agent generally tend to vary with concentration, the degree of variation depends on the type of cell, concentration, and so on, and thus the concentration ranges described above represent general trends.

“Alginic acid” includes pharmaceutically acceptable salts of alginic acid. Such a pharmaceutically acceptable salt of alginic acid is such that a hydrogen ion of the carboxylic group of the alginic acid is liberated and a cation is bound instead of the hydrogen ion. Such a cation may be any cation capable of forming a pharmaceutically acceptable salt, and examples of such a cation include monovalent cations such as sodium ion, potassium ion, and ammonium ion, and multivalent cations including inorganic multivalent ions such as calcium ion, magnesium ion, iron ion, and ammonium ion, and organic multivalent ions such as polylysine.

For such alginic acid, a commercially available product can be used.

The word “heparins” means heparin and heparinoid. Heparin is one of glycosaminoglycans that are produced mainly in the liver in a living body. An average molecular weight is known to generally range from 3000 to 35000. Heparins are N-sulfate, N-acetyl and O-sulfate substitutes of linear polysaccharide in which uronic acid (D-glucuronic acid or L-isuronic acid) and D-glucosamine are alternately bound, are known to generally have three sulfate groups per disaccharide, and are most sulfated acidic polysaccharides. Heparin has anti-coagulant activity, lipemic clearing activity, and so on. Heparinoid is obtained by modifying and partially degrading heparin, and has the same physiological activity as heparin. Examples of heparinoid include a salt of heparin capable of forming a pharmaceutically acceptable salt (for example, alkali metal salts, alkali earth metal salts, etc.), unfractionated heparin, low molecular weight heparin, danaparoid, and salts thereof, and an anti-clotting agent such as fondaparinux. Examples of a salt of heparin include a sodium salt, a potassium salt, and an ammonium salt. Low molecular weight heparin preferably has a number average molecular weight of 2000 to 8000. As heparins, commercially available products can be used.

Dextran sulfate (hereinafter, sometimes referred to as DS) is dextran sulfate or a pharmaceutically acceptable salt thereof. A pharmaceutically acceptable salt of dextran sulfate is such that a hydrogen ion of the sulfonic group of dextran sulfate is liberated and a cation is bound in stead of the hydrogen ion. Such a cation may be any cation capable of forming a pharmaceutically acceptable salt, and examples of such a cation include monovalent cations such as sodium ions, potassium ions, and ammonium ions. An average molecular weight (Mw) of dextran sulfate preferably ranges from 200 to 1000000. A sulfur content is preferably 0.00001 to 2, and more preferably 0.001 to 2, as the number of bound sulfate groups per one monosaccharide. The average molecular weight (Mw) and the sulfur content can be measured in accordance with the methods described in the Japanese Pharmacopoeia. As dextran sulfate, commercially available products can be used.

A proteolytic enzyme inhibitor (hereinafter, sometimes simply referred to as “inhibitor”) is also called a protease inhibitor, and commercially available various products can be used without particular limitation. Examples of such a proteolytic enzyme inhibitor include, but are not limited to, urinastatin, benzamidine, phenylmethylsulfonyl fluoride (PMSF), 4-(2-aminoethyl)benzene fluoride (AEBSF), aprotinin, E-64, ethylenediaminetetraacetic acid (EDTA), glycol ether diamine tetraacetic acid (EGTA), leupeptin, leupeptin hemisulfate, antipain, chymostatin, pepstatin A, phosphoramidon, bestatin, sibelestat sodium hydrate, fosamprenavir calcium hydrate, darunavir ethanol adduct, lopinavir, ritonavir, aprotinin and pharmaceutically acceptable salts thereof, and nafamostat mesylate, alafenamide fumarate, camostat mesylate, atazanavir sulfate, and so on. They may be used alone or in combination of two or more kinds.

Alginic acid, heparins, dextran sulfate, and a proteolytic enzyme inhibitor may be used alone or in combination of two or more kinds.

A dosage form of the cell treatment agent is not particularly limited, and can be appropriately determined as powder, liquid, or the like using an appropriate excipient according to various applications. Also, if necessary, various additives can be added.

For example, when desired cells are cultured in a general cell culture solution and then the cells are administered into a body, the cells are separated from the cell culture solution, and washed to remove various reagents that are not used for medical purposes and contained in the cell culture solution, and then the obtained cells are immersed in an extracellular fluid replacement solution, a maintenance infusion, or the like, and can be administered to the body. The extracellular fluid replacement solution is a group of liquids having an electrolyte composition similar to the extracellular fluid surrounding cells in a living body, and the maintenance infusion is an infusion solution that is administered while nutrients such as sugars, proteins (amino acids), and fats and micronutrients are added to the daily water content and the electrolyte required for humans to maintain life. The extracellular fluid replacement solution and the maintenance infusion are medical liquids used as solvents for injection solutions of other active ingredients or for intravenous drip injection of itself alone, and the safety of administration into bodies has been established. Thus, for the electrolyte composition similar to the environment surrounding cells and safety, an extracellular fluid replacement solution or a maintenance infusion is suitable as an excipient of a cell treatment agent used in administering cells into a body in regenerative medicine. Examples of the extracellular fluid replacement solution include so-called replacement infusions used for the purpose of replenishing the loss of extracellular fluid, and more specific examples include a Ringer's solution, a lactated Ringer's solution, an acetated Ringer's solution, a bicarbonate Ringer's solution, a Hartmann solution, saline, a plasma substitute, a plasma preparation, and the like. Among these, for example, replacement infusions that are not derived from human, such as a plasma substitute and a plasma preparation, are desirable. Examples of the maintenance infusion include: a sugar and electrolyte infusion preparation that does not contain amino acid; a sugar, electrolyte and amino acid infusion preparation; a sugar, electrolyte, amino acid, and multivitamin liquid preparation; a sugar, electrolyte, amino acid, multivitamin, and trace element liquid preparation; and a sugar, electrolyte, amino acid, and fat emulsion; and the like.

Meanwhile, when cells are separated from a general cell culture solution and washed, and then immersed in a so-called extracellular fluid replacement solution to prepare a cell treatment agent as described above, these operations may reduce the viability of cells or cause infection. Meanwhile, the present inventor has found that the active ingredient of the treatment agent has a cell protective effect in the extracellular fluid replacement solution as well as in a general cell culture solution. That is, an extracellular fluid replacement solution, a maintenance infusion, or the like contains the active ingredient of the treatment agent so as to be used as a substitute for a cell culture solution, for example, in preservation and transportation, and in administration, the cells can be immediately administered while they are immersed in the extracellular fluid replacement solution. Therefore, it is possible to prevent the decrease in viability and the risk of infection caused by transferring cells from the culture solution to a so-called extracellular fluid replacement solution, maintenance infusion, or the like.

The above-described cell treatment agent can be used as a reagent in a predetermined dosage form containing the above-described specific active ingredient. Therefore, by adding or applying the reagent to a culture substrate such as a culture solution, a culture vessel, and a carrier of a cell, or to a living organ, it is possible to (a) detach cultured cells grown on a scaffold of the culture substrate or on a scaffold of the living tissue from the scaffold of the culture substrate or the scaffold of the living tissue and suspend the same, and to (b) detach cells in tissue of the living tissue from the scaffold (living organ, etc.) of the living tissue and suspend the same. Therefore, the cell treatment agent can be suitably used for cell detachment and suspension.

For cells in tissue of a living tissue, or cultured cells on the scaffold of the culture substrate or on the scaffold of the living tissue, the above-described cell treatment agent can stop proliferation, stop adhesion, or maintain an undifferentiated or low differentiated state without allowing differentiation of the cells while keeping their viability, in other words, can make the cells dormant. Therefore, the cell treatment agent can be suitably used for cell dormancy for making these cells dormant. Owing to this dormancy effect, cells stop proliferating, but are in such a state that they awake and re-adhere to the culture substrate or the living tissue and start proliferation when the conditions are satisfied. Thus, the cell treatment agent is effective in the point of being capable of temporarily making cells dormant, stopping activities of the cells for preserving and transporting the cells, and resuming the activities at a desired timing, and in the point of being capable of directly making suspended cells dormant, which are obtained by a detachment and suspension treatment. In addition, since the above-described cell treatment agent is capable of maintaining cells in an undifferentiated or low differentiated state, it is also suited for cell preservation that enables a tissue or an organ exhibiting desired properties to be regenerated or formed after awakening of the cells from the dormant state.

The above-described cell treatment agent has a cell protective effect of maintaining viability of cells in tissue or cultured cells in a cell container including a culture vessel, or in a living tissue to prevent death of the cells. Therefore, the cell treatment agent can be used as a substitute for FBS or human serum, which has been conventionally used for having the protective effect of preventing cell death. Therefore, it is possible to safely prevent cell death and protect cells without using FBS or human serum. The cell treatment agent having the cell protective effect is suitable, for example, for preserving and transporting cells. In particular, as described above, the cell treatment agent containing an extracellular fluid replacement solution or a maintenance infusion solution as an excipient, and the above-described specific active ingredient can protect cells even during preservation and transportation, and can be administered directly as it is. Therefore, the cell treatment agent greatly contributes to the safety and convenience of medical care.

Since the cell treatment agent can function, for example, for cell dormancy, for cell protection, or for cell preservation for maintaining cells in an undifferentiated or low differentiated state as described above, the cell treatment agent in a liquid dosage form is suitable as a liquid for the preservation of cells, tissues, or organs. In this case, the dosage form is preferably a cell culture solution, an extracellular fluid replacement solution, a maintenance infusion, or the like.

By using the cell treatment agent as described above, cells can be made dormant, and by making a preparation containing multivalent cations act on the dormant cells, the dormant cells can be awakened. Examples of such preparations containing multivalent cations include: aqueous solutions containing polycations such as chitosan, or polyvalent cations such as aluminum ions, iron ions, magnesium ions, or calcium ions; chelated preparations of these polyvalent cations; solids that generate insoluble polyvalent cations such as powder of aluminum compounds, iron compounds, magnesium compounds, or calcium compounds; and cloth pieces in which these polyvalent cation suppliers are impregnated or carried on a surface. Examples of a calcium ion source include calcium chloride, calcium gluconate, calcium carbonate, and calcium phosphate; examples of an iron ion source include iron hydroxide; and examples of an aluminum ion source include aluminum hydroxide. Examples of a multivalent cation supplier include chelated multivalent cations. Examples of the fabric include gauze, made of biocompatible fibers, that is applicable to a living body. A use amount of a preparation containing multivalent cations can be determined according to the target of application, the composition of the active ingredient of the treatment agent, the form of the preparation, and so on. As described above, by combining the cell treatment agent and the preparation containing multivalent cations, it is possible to provide a set reagent capable of awakening dormant cells.

The adding amount of the cell treatment agent in various applications can be appropriately determined according to the application target, the composition of the active ingredient of the treatment agent, and the like. In application to a culture solution for cell protection, the cell treatment agent can be added so that the concentration of alginic acid is 200 to 0.001 mg/ml, the concentration of dextran sulfate is 100 to 0.0001 mg/ml, and the concentration of heparins is 1000 to 0.001 units/ml, for example. The inhibitor can be appropriately determined according to the kind thereof or the like, and for example, the cell treatment agent can be added so that the concentration of urinastatin is 2500 to 0.01 units/ml, the concentration of nafamostat mesylate is 5 to 0.00001 mg/ml, and the concentration of gabexate mesylate is 5 to 0.00001 mg/ml.

### EXAMPLES

Hereinafter, embodiments of the present invention will be described in detail on the basis of examples. In the examples shown below, various effects on cells in a cell culture vessel or storage vessel are verified; however, it goes without saying that the same effects are also exhibited on cells in living tissues, spheroid cells obtained by three-dimensional cell culture, or the like.

(Cells Used)

Experiments were performed using Chinese hamster fibroblasts as mesenchymal cells, rat corneal epithelial cells as epithelial cells, human amnion-derived mesenchymal stem cells as cells in an undifferentiated state, and a highly metastatic strain of mouse malignant melanoma as malignant tumor.

(Preparation of Cells)

The aforementioned cells were cultured in a culture vessel under normal cell culture conditions (37° C., humidity of 100%, CO₂ concentration of 5%) in an FBS-added cell culture solution prepared by adding 10% FBS to a normal serum-free cell culture solution. Cells in the culture vessel were then detached and suspended by the ordinary trypsin method. The obtained detached and suspended cells were used in the following experimental examples. The ordinary trypsin method was performed as follows.

The cell culture solution in the cell culture vessel was sucked out, and the cell surface was washed with about half of the amount for use in culture of PBS (−) (PBS without Ca²⁺/Mg²⁺). Thereafter, 1 mL of 0.2% trypsin/EDTA solution was distributed in the vessel with respect to the cell surface of 25 cm² to let trypsin act. Then the excess trypsin solution was removed. The culture vessel was incubated in a CO₂ incubator for 2 minutes under normal conditions (37° C., CO₂ concentration of 5%, humidity of 100%), and cell detachment from the inner wall surface of the culture vessel was examined. Trypsin was then completely inactivated with a trypsin inhibitor in the case of using a serum-free cell culture solution. In the case of using an FBS-added cell culture solution, an FBS-added cell culture solution was added to completely inactivate trypsin.

### Experimental Example 1: Cell Protective Effect

(1-1) Alginic Acid

(1-1-1) Preservation at Normal Temperature for 24 Hours

Using a normal serum-free cell culture solution without FBS or a Ringer's solution (Otsuka Pharmaceutical Co., Ltd., Lactec Injection, Japanese Pharmacopoeia, Sodium L-lactate Ringer's solution) as a conditioning solution for use, for example, in preservation and transportation of cells, sodium alginate (Na alginate) (Fuji Chemical Industry Co., Ltd. Snow Algin SSL, viscosity of 1% aqueous solution is 30 mPa·s at 20° C.) was added to the conditioning solution in the concentrations shown in Table 1 to prepare cell preservation solutions. To each cell preservation solution, the above-described cells were added at a concentration of 10⁶ cells/ml to satisfy the conditions shown in Table 1, and the resultant solution was left to stand in a room at normal temperature (22° C.) for 24 hours, and then whether cells were alive or dead was determined, and rates of viable cells (average survival rate (%)) were calculated and compared. Cell viability was determined by the trypan blue excretion test that is the ordinary method of confirming cell viability. The excretion test was performed according to the method described in “Denizot F., et al., Rapid colorimetric assay for cell growth and survival. Modifications to the tetrazolium dye procedure giving improved sensitivity and reliability. J. Immunol. Methods, 89, 271-277 (1986)”. The results are shown in Table 1. The results are of N=4.

TABLE 1

| | | Concentration of Na | Average survival |
|---|---|---|---|
| | Conditioning | alginate | rate at |
| Cell | solution | (mg/mL) | 24 hours (%) |
| Rat corneal | Serum-free cell | 0 | 46 |
| epithelial cell | culture solution | 0.63 | 60 |
| | | 2.5 | 70 |
| | | 5 | 74 |
| | Ringer's | 0 | 36 |
| | solution | 0.63 | 52 |
| | | 1.25 | 51 |
| | | 2.5 | 53 |
| Chinese | Ringer's | 0 | 39 |
| hamster | solution | 0.63 | 56 |
| fibroblast | | 1.25 | 57 |
| | | 2.5 | 60 |

(1-1-2) Preservation Under Refrigeration for 72 Hours

The average survival rate (%) was determined in the same manner as in Experimental Example (1-1-1) except that the preservation conditions were changed to leaving still for 72 hours in a refrigerator at a set temperature of 4° C. The results are shown in Table 2. The results are of N=4.

TABLE 2

| | | Concentration of Na | Average survival |
|---|---|---|---|
| | Conditioning | alginate | rate at |
| Cell | solution | (mg/mL) | 72 hours (%) |
| Rat corneal | Serum-free cell | 0 | 62 |
| epithelial cell | culture solution | 0.63 | 77 |
| | | 2.5 | 80 |
| | | 5 | 70 |
| | Ringer's | 0 | 55 |
| | solution | 0.63 | 68 |
| | | 2.5 | 72 |
| | | 5 | 64 |
| Chinese | Ringer's | 0 | 61 |
| hamster | solution | 0.63 | 73 |
| fibroblast | | 2.5 | 77 |
| | | 5 | 70 |

As shown in Tables 1 and 2, cell preservation solutions containing alginic acid showed superior cell viability compared to a serum-free cell culture solution or a Ringer's solution without alginic acid under preservation conditions of 24 hours at normal temperature and 72 hours under refrigeration (4° C.), indicating that they have excellent cell protective effects. The effect of improving cell viability by addition of alginic acid, namely, the cell protective effect was also observed in rat corneal epithelial cells, which are epithelial cells, and in Chinese hamster fibroblasts, which are mesenchymal cells.

(1-2) Dextran Sulfate

(1-2-1) Preservation at Normal Temperature for 24 Hours

The average survival rate (%) of cells was determined in the same manner as in Experimental Example (1-1-1) except that dextran sulfate (dextran sulfate special grade reagent available from NACALAI TESQUE, INC.) was used instead of sodium alginate and the conditions were as shown in Table 3. The results are shown in Table 3. The results are of N=4.

TABLE 3

| | | | Average survival |
|---|---|---|---|
| | Conditioning | DS concentration | rate at |
| Cell | solution | (mg/mL) | 24 hours (%) |
| Rat corneal | Serum-free cell | 0 | 46 |
| epithelial cell | culture solution | 0.003 | 62 |
| | | 0.1 | 71 |
| | | 1 | 73 |

(1-2-2) Preservation Under Refrigeration for 72 Hours

The average survival rate (%) of cells was determined in the same manner as in Experimental Example (1-2-1) except that the preservation conditions were changed to leaving still for 72 hours in a refrigerator at a set temperature of 4° C. and to conditions shown in Table 4. The results are shown in Table 4. The results are of N=3 in the case of using a serum-free cell culture solution, and are of N=4 in the case of using a Ringer's solution.

TABLE 4

| | | | Average survival |
|---|---|---|---|
| | Conditioning | DS concentration | rate at |
| Cell | solution | (mg/mL) | 72 hours (%) |
| Rat corneal | Serum-free cell | 0 | 62 |
| epithelial cell | culture solution | 0.003 | 76 |
| | | 0.1 | 79 |
| | | 1 | 73 |
| | Ringer's | 0 | 55 |
| | solution | 0.003 | 67 |
| | | 0.1 | 71 |
| | | 1 | 60 |

As shown in Tables 3 and 4, cell preservation solutions containing dextran sulfate showed superior cell viability compared to cell preservation solutions without dextran sulfate under preservation conditions of 24 hours at normal temperature and 72 hours under refrigeration (4° C.) irrespective of the conditioning solution used, indicating that they have excellent cell protective effects. The effect of improving cell viability by addition of dextran sulfate, namely, the cell protective effect was also observed for Chinese hamster fibroblasts, which are mesenchymal cells.

(1-3) Heparins

(1-3-1) Preservation at Normal Temperature for 24 Hours

The average survival rate (%) of cells was determined in the same manner as in Experimental Example (1-1-1) except that heparin sodium (Heparin Na Mochida, unfractionated heparin, available from Mochida Pharmaceutical Co., Ltd.) was used instead of alginic acid and the conditions were as shown in Table 5. The results are shown in Table 5. The results are of N=4.

TABLE 5

| | | Heparin Na | Average survival |
|---|---|---|---|
| | Conditioning | concentration | rate at |
| Cell | solution | (units/mL) | 24 hours (%) |
| Rat corneal | Serum-free | 0 | 46 |
| epithelial | cell culture | 5 | 69 |
| cell | solution | 25 | 75 |
| | | 125 | 76 |

(1-3-2) Preservation Under Refrigeration for 72 Hours

The average survival rate (%) of cells was determined in the same manner as in Experimental Example (1-3-1) except that the preservation conditions were changed to leaving still for 72 hours in a refrigerator at a set temperature of 4° C. and to conditions shown in Table 6. The results are shown in Table 6. The results are of N=4.

TABLE 6

| | | Heparin Na | Average survival |
|---|---|---|---|
| | Conditioning | concentration | rate at |
| Cell | solution | (units/mL) | 72 hours (%) |
| Rat corneal | Ringer's | 0 | 55 |
| epithelial | solution | 5 | 70 |
| cell | | 25 | 72 |
| | | 125 | 74 |

As shown in Tables 5 and 6, cell preservation solutions containing heparin sodium showed superior cell viability compared to cell preservation solutions without heparin sodium (namely, only a conditioning solution) under preservation conditions of 24 hours at normal temperature and 72 hours under refrigeration (4° C.) irrespective of the conditioning solution used, indicating that they have excellent cell protective effects. The effect of improving cell viability by addition of heparin sodium, namely, the cell protective effect was also observed for Chinese hamster fibroblasts, which are mesenchymal cells. Further, also when the same experiment was conducted using low molecular weight heparin sodium instead of heparin sodium, the same cell viability improving effect, namely, the cell protective effect was observed for each cell.

(1-4) Proteolytic Enzyme Inhibitor

(1-4-1) Ulinastatin (Inhibitor a)

(1-4-1-1) Preservation Under Refrigeration for 72 Hours

The average survival rate (%) of cells was determined in the same manner as in Experimental Example (1-3-2) except that urinastatin (Miraclid injection solution, containing 2.5 million units, available from Mochida Pharmaceutical Co., Ltd.) was used as a proteolytic enzyme inhibitor instead of heparin sodium and the conditions were as shown in Table 7. The results are shown in Table 7. The results are of N=4. “Unit (U)” in the unit of concentration (units/mL) of inhibitor a in Table 7 can be measured, for example, by the quantitative method of urinastatin in the Japanese Pharmacopoeia 15th revision. Specifically, absorbance at 405 nm of a sample solution is measured with a UV 240 spectrophotometer, and concentration of urinastatin can be calculated from a calibration curve prepared in advance.

TABLE 7

| | | Concentration of | Average |
|---|---|---|---|
| | Conditioning | inhibitor | survival rate |
| Cell | solution | a (units/mL) | at 72 hours (%) |
| Rat corneal | Ringer's | 0 | 55 |
| epithelial cell | solution | 5 | 70 |

(1-4-2) Nafamostat Mesilate (Inhibitor b)

(1-4-2-1) Preservation Under Refrigeration for 72 Hours

Using a 5% glucose solution as a conditioning solution, nafamostat mesilate (Fusan for Injection, available from Nichi-Iko Corporation), which is a proteolytic enzyme inhibitor, was added to the conditioning solution in the concentrations shown in Table 8 (in Fusan for Injection (available from Nichi-Iko Corporation), use of a 5% glucose solution as a dilution solution is specified) to prepare cell preservation solutions. To each cell preservation solution, cells of mouse malignant melanoma high metastatic strain were added at a concentration of 10⁶ cells/ml and left in a refrigerator set at 4° C. for 48 hours, and then the average survival rate (%) of cells was determined in the same manner as in Experimental Example of (1-4-1-1). The results are shown in Table 8. The results are of N=4.

TABLE 8

| | | Concentration of | Average survival |
|---|---|---|---|
| | Conditioning | inhibitor | rate at |
| Cell | solution | b (mg/mL) | 48 hours (%) |
| Mouse | 5% glucose | 0 | 30< |
| malignant | solution | | |
| melanoma | | | |
| highly | | 0.25 | 43 |
| metastatic | | | |
| strain cell | | | |

(1-4-3) Mixture of Protease Inhibitor (Inhibitor c)

(1-4-3-1) Preservation Under Refrigeration for 72 Hours

The average survival rate (%) of cells was determined in the same manner as in Experimental Example (1-4-1-1) except that a mixture of protease inhibitor (protease inhibitor cocktail set I, available from FUJIFILM Wako Pure Chemical Corporation) was used instead of urinastatin and the conditions were as shown in Table 9. The results are shown in Table 9. The results are of N=4. The mixture of protease inhibitor contains 50 mmol/l of AEBSF hydrochloride, 15 μmol/l of aprotinin (recombinant), 0.1 mmol/l of E-64, 50 mmol/l of EDTA·2Na·2H₂O, and 0.1 mmol/l of leupeptin hemisulfate when one vial is dissolved in 1 ml distilled water. This was used as an undiluted solution and added to the conditioning solution to provide the dilution ratios shown in Table 9.

TABLE 9

| | | Dilution ratio of | Average |
|---|---|---|---|
| | Conditioning | inhibitor c against | survival rate |
| Cell | solution | undiluted solution | at 72 hours (%) |
| Rat corneal | Ringer's | Not contained | 53 |
| epithelial cell | solution | Diluted 200 times | 69 |
| | | Diluted 100 times | 73 |

As shown in Tables 7 to 9, cell preservation solutions containing a proteolytic enzyme inhibitor showed superior cell viability compared to cell preservation solutions without a proteolytic enzyme inhibitor (namely, only a conditioning solution) under preservation conditions of 72 hours or 48 hours under refrigeration (4° C.), indicating that they have excellent cell protective effects. The effect of improving cell viability by addition of a proteolytic enzyme inhibitor, namely, the cell protective effect was also observed for Chinese hamster fibroblasts, which are mesenchymal cells.

### Experimental Example 2: Cell Dormancy Effect

Sodium alginate (sodium alginate first grade reagent, available from FUJIFILM Wako Pure Chemical Corporation, viscosity of 1% aqueous solution is 120 mPa·s at 20° C.), DS (the same as in Experimental Example 1), or sodium heparin (the same as in Experimental Example 1) was used as an active ingredient in the cell treatment agent, and whether cell dormancy occurs or not by action of the active ingredient was examined by comparing the activities of cell division and proliferation which are the most general cell activities in the following manner.

(2-1) Introduction to Cell Dormancy

The cell cycle was analyzed by flow cytometry according to the method described in known literature (Piotr Pozarowski and Zbigniew Darzynkiewicz, “Analysis of Cell Cycle by Flow Cytometry,” Methods in Molecular Biology, vol. 281: Checkpoint Controls and Cancer, Volume 2), and frequency of cell division and proliferation was compared between different active ingredients of the cell treatment agent. A specific method is as follows. Using a normal serum-free cell culture solution as a conditioning solution (comparison reference (a)), (b) a cell preservation solution in which sodium alginate (Na alginate) is added to the conditioning solution in a concentration of 2.5 mg/ml, (c) a cell preservation solution in which DS is added to the conditioning solution in a concentration of 0.1 mg/ml, and (d) a cell preservation solution in which heparin sodium (heparin Na) is added to the conditioning solution in a concentration of 50 units/ml, were prepared. To these cell preservation solutions, rat corneal epithelial cells were added at a cell concentration of 10⁵ cells/ml and cultured for 48 hours under normal cell culture conditions (37° C., CO₂ concentration of 5%, humidity of 100%). The number of cells in each culture was determined by cell cycle analysis using flow cytometry to determine the number of cells in each cell cycle, and the percentage of cells in the S phase, G2 phase, and M phase, which are considered as the periods of the cell division and proliferation process, was compared with the percentage of cells in the G0 phase and G1 phase, which are considered as other periods. The results are shown in Table 10. The results are of N=4.

TABLE 10

| | | | Percentages of cells | |
|---|---|---|---|---|
| | | | of each cell cycle | |
| | | Concentration | | S phase + |
| | Conditioning | of active | G0 phase + | G2 phase + |
| Cell | solution | ingredient | G1 phase | M phase |
| Rat corneal | Serum-free | — | 76.7 | 22.7 |
| epithelial | cell culture | Na alginate | 88.1 | 10.1 |
| cell | solution | 2.5 mg/mL | | |
| | | DS | 92.4 | 5.77 |
| | | 0.1 mg/mL | | |
| | | Heparin Na | 89.7 | 7.68 |
| | | 50 units/mL | | |

As shown in Table 10, the percentage of cells in the S phase, G2 phase, and M phase, which are considered as the periods of the cell division and proliferation process, was significantly reduced when each ingredient was added, compared to the serum-free cell culture solution alone, which was used as a comparison reference. In other words, when each ingredient is added to the serum-free cell culture solution, the cell division and proliferation functions are reduced and the cells become dormant. Therefore, it can be seen that the cell preservation solution to which each ingredient is added is effective as a cell treatment agent for cell dormancy that makes cultured cells dormant.

(2-2) Differentiation State Maintenance

Using dextran sulfate (DS) as an active ingredient of the cell treatment agent, the cell dormancy effect by DS was considered according to the effect of preventing differentiation by maintaining the cells in an undifferentiated state, as an index. As the cells, human amnion-derived mesenchymal stem cells in an undifferentiated state were used. Using a normal serum-free cell culture solution as a conditioning solution, a cell preservation solution a was prepared by adding DS to the conditioning solution at 0.1 mg/ml, and a cell preservation solution b was prepared by adding FBS to the conditioning solution at 10%. To each of the cell preservation solutions a and b, the aforementioned stem cells were added at 10⁵ cells/ml, respectively, and then cultured for 5 days under normal cell culture conditions (37° C., CO₂ concentration of 5%, humidity of 100%). The degrees of differentiation of stem cells before culture and after 5 days of culture were evaluated and compared by flow cytometry. The method followed the method described in Masoumeh Fakhr Taha, Vahideh Hedayati “Isolation, identification and multipotential differentiation of mouse adipose tissue-derived stem cells” Tissue and Cell 42 (2010) 211-216. The evaluation was made according to CD29 and CD44 as undifferentiation markers and CD11b, CD31, and CD45 as markers of differentiation into mesenchymal cells. The results are shown in Table 11.

TABLE 11

| | | | After culture | |
|---|---|---|---|---|
| | Marker | Before culture | DS 0.1 mg/mL | 10% FBS |
| Positive | CD29 | 98.8 | 98.7 | 98.5 |
| rate of | CD44 | 62.3 | 62.3 | 62.7 |
| marker (%) | CD11b | 16.6 | 19.8 | 48.2 |
| | CD31 | 7.9 | 8.3 | 32.5 |
| | CD45 | 7.0 | 7.3 | 19.6 |

As shown in Table 11, when a serum-free cell culture solution containing DS was used as a cell preservation solution, the positive rates of all three differentiation markers increased only slightly after culture, compared to before culture, resulting in that the cells remained undifferentiated. In contrast, when a serum-free cell culture solution containing FBS was used as a cell preservation solution, the positive rates of all three differentiation markers increased significantly after culture, compared to before culture, revealing that the cells began to change in the direction of cell differentiation. This reveals that DS prevents differentiation by making cells in an undifferentiated state dormant, while FBS also having the cell protective effect does not prevent differentiation of cells.

### Experimental Example 3: Awakening from Cell Dormancy

Whether or not cells having been made dormant by the action of sodium alginate (the same as in Experimental Example 2), DS (the same as in Experimental Example 1), or heparin sodium (the same as in Experimental Example 1) used as an active ingredient of the cell treatment agent, as shown in the aforementioned experimental examples, were awakened by a preparation containing calcium ions (Carticol injection solution, available from Nichi-Iko Pharmaceutical Co., Ltd., calcium gluconate hydrate solution) was examined, according to whether cell division and proliferation which are the most general cell activities of cells resume by addition of a calcium agent as an index.

(3-1) Awakening from Cell Dormancy

Using a normal serum-free cell culture solution as a conditioning solution ((a) comparison reference), (b) a cell preservation solution in which DS is added to the conditioning solution in a concentration of 0.1 mg/ml, (c) a cell preservation solution in which heparin sodium (heparin Na) is added to the conditioning solution in a concentration of 50 units/ml, and (d) a cell preservation solution in which sodium alginate (Na alginate) is added to the conditioning solution in a concentration of 2.5 mg/ml were prepared. Next, rat corneal epithelial cells obtained as described above were added to the cell preservation solutions (a) to (d) to provide a cell count of 1×10⁵ cells/ml, and these were stored under refrigeration at 4° C. for 48 hours. The sample numbers of the cells contained in the cell preservation solutions (a) to (d) are denoted by (a′) to (d′), respectively.

Cell preservation solutions (a) to (d) were separately prepared in the same manner, and these were divided into two groups, with sample numbers a-1 to d-1 and sample numbers a-2 to d-2, respectively. Nothing was added to the cell preservation solutions of sample numbers a-1 to d-1, and a calcicol injection solution was added to the cell preservation solutions of sample numbers b-2 to d-2 to provide a calcium concentration of 5.23 mg/ml.

Cell preservation solutions (a) to (d) containing cells after 48 hours of preservation under refrigeration, which were obtained as described above, were centrifuged, and cells (a′) to (d′) were collected by centrifugation, respectively, and the cells (a′) to (d′) were added to cell preservation solutions a-1 to d-2 with the corresponding alphabetical sample numbers so that the cell content was 6×10⁴ cells/ml, respectively.

Cell preservation solutions a-1 to d-2 to which predetermined cells (a′) to (d′) were added were cultured for 120 hours under normal cell culture conditions (37° C., CO₂ concentration of 5%, humidity of 100%).

After culturing, the number of viable cells per unit volume in each of cell preservation solutions a-1 to d-2 was quantified by the MTT assay. The MTT assay method was performed according to known literature (Priti Kumar et al. “Analysis of Cell Viability by the MTT Assay” Cold Spring Harbor Protocols. 6; 2018 DOI: 10.1101/pdb.prot095505). Culturing conditions and measurement results are shown in Table 12. The results are of N=12.

TABLE 12

| | | | | | | Average content of | |
|---|---|---|---|---|---|---|---|
| | | | | | | viable cells | |
| | | | | | | (×10⁴ cells/ml) | |
| Sample number | | Concentration of ingredient* added to serum- | | | | At the | |
| of cell | Sample | free cell culture solution | | | | time of | After 120 |
| preservation | number of | DS | Heparin | Na alginate | Calcium | cell | hours of |
| solution | cell | (mg/mL) | (units/mL) | (mg/mL) | (mg/mL) | addition | culture |
| a-1 | (a′) | — | — | — | — | 6 | 19.1 |
| b-1 | (b′) | 0.1 | — | — | — | 6 | 11 |
| b-2 | | 0.1 | — | — | 5.23 | 6 | 24.3 |
| c-1 | (c′) | — | 50 | — | — | 6 | 14.2 |
| c-2 | | — | 50 | — | 5.23 | 6 | 25.6 |
| d-1 | (d′) | — | — | 2.5 | — | 6 | 12.1 |
| d-2 | | — | — | 2.5 | 5.23 | 6 | 27.5 |

*excluding ingredient initially contained in serum-free cell culture solution

As shown in Table 12, when calcium ions are not added, the increase in the number of viable cells is suppressed in the cases (b-1 to d-1) containing each ingredient used as an active ingredient in the cell treatment agent, compared to the case (a-1) not containing the same. On the other hand, when calcium ions are added (b-2 to d-2), the number of viable cells increases. The same experiment was performed using Chinese hamster fibroblasts, and equivalent results were obtained. These experiments revealed that by containing each ingredient used as an active ingredient of the cell treatment agent, the reduced division and proliferation ability of dormant cells are inversely activated due to awakening of cells by addition of calcium ions.

### Experimental Example 4: Cell Detaching and Suspending Effect

<Preparation of Adherent Cells>

A culture solution in which FBS was added to a normal serum-free cell culture solution in a concentration of 10% (10% FBS cell culture solution) was prepared in a culture dish. To this, rat corneal epithelial cells were added at 10⁶ cells/ml and cultured for 24 hours under normal cell culture conditions (37° C., CO₂ concentration of 5%, humidity of 100%) to make the cultured cells fully adhere to the culture dish wall which is a scaffold.

<Detachment and Suspension of Adherent Cells>

The culture solution in the culture dish on which cultured cells obtained as described above had adhered was replaced with each of dextran sulfate aqueous solutions and a dextran aqueous solution, which are respectively obtained by dissolving dextran sulfate (Dextran sodium sulfate MW 36000-50000, available from FUJIFILM Wako Pure Chemical Corporation) and dextran (Dextran MW 35000-50000, available from FUJIFILM Wako Pure Chemical Corporation) in purified water and have concentrations shown in Table 13, and the resultant culture solutions were continuously cultured under normal cell culture conditions (37° C., CO₂ concentration of 5%, humidity of 100%). For each adherent cell, detachment and suspension of adherent cells from the dish wall was observed over time. For determination of detachment and suspension, the culture dish was gently shaken manually to observe whether the cells were detached from the dish wall and suspended, and the case where 90% or more of the cells were detached and suspended was determined as detachment and suspension of adherent cells. The results for the cases of the dextran sulfate aqueous solution are shown in Table 13. The results are of N=4. In Table 13, “∘” means that the cells were detached and suspended, and “x” means that the cells were not detached and suspended. In the case of replacement with the dextran aqueous solution, the adherent cells were not detached and suspended in any of the cases.

TABLE 13

| DS | | | | |
|---|---|---|---|---|
| concentration | Lapse time after replacement (hour) | | | |
| (mg/mL) | <12 | 12 | 24 | 48 |

| | | |
|---|---|---|
| 0.1 | x | ∘ |

| | | |
|---|---|---|
| 1 | x | ∘ |

| | | |
|---|---|---|
| 10 | x | ∘ |

As shown in Table 13, when DS was contained, the adherent cells having adhered to the inner wall of the culture dish were detached and suspended within a predetermined time depending on the concentration of DS. The same experiment was performed using Chinese hamster fibroblasts, and equivalent results were obtained. Thus, it was found that, unlike normal dextran, DS has a cell detaching and suspending effect that detaches and suspends adherent cells.

### Experimental Example 5: Cell Detaching and Suspending Effect and Cell Re-Adhering Effect

<Preparation of Adherent Cells>

In the same manner as in Experimental Example 4, rat corneal epithelial cells were sufficiently adhered to the culture dish wall which is a scaffold.

<Detachment and Suspension of Adherent Cells>

To the culture solutions in the culture dish on which cultured cells obtained in the manner as described above had adhered, each active ingredient was added in a concentration shown in Table 14, or no active ingredient was added. Each culture dish was then placed on the base of a shaker (product name: Laboshaker Model BC-740, available from BIO CRAFT Corporation), the shaking intensity was set to the lowest level, and the shaking frequency was set to 4 times/min, and cells were cultured under normal cell culture conditions (37° C., CO₂ concentration of 5%, humidity of 100%) for 24 hours while each culture dish was gently shaken. Then, detachment and suspension of cells after 24 hours of culturing was observed. For determination of detachment and suspension, whether cells were detached from the dish wall and suspended under shaking of the culture dish was observed, and the case where 90% or more of the cells were detached and suspended was determined as “detached and suspended”, and “detached and suspended” cells were marked with “∘” and “undetached and unsuspended” cells were marked with “x”. The results are shown in Table 14. The results are of N=4.

TABLE 14

| | | | Detachment at |
|---|---|---|---|
| | | | lapse of 24 |
| | | Concentration of | hours after |
| | Conditioning | active | addition of |
| Cell | solution | ingredient | active ingredient |
| Rat corneal | Cell culture | — | x |
| epithelial | solution | | |
| cell | containing 10% | Heparin Na | ∘ |
| | FBS | 100 units/mL | |
| | | DS | ∘ |
| | | 0.5 mg/mL | |

<Re-Adhesion of Detached and Suspended Cells>

Whether the cells having been detached and suspended as described above were re-adhered by calcium ions was examined. Part of the bottom of the culture dish was coated with a slurry of calcium phosphate fine powder mixed with distilled water and dried in an oven to separately prepare a culture dish in which a film of fine powder of calcium phosphate was stuck to the bottom of the dish. Then, to the culture dish with the calcium phosphate film and the culture dish without the film (control), a cell culture solution containing the detached and suspended cells as described above was added, the culture dishes were placed on the base of a shaker (product name: Laboshaker Model BC-740, available from BIO CRAFT Corporation), the shaking intensity was set to the lowest level, the shaking frequency was set to 4 times/min, and the cells were cultured under normal cell culture conditions (37° C., CO₂ concentration of 5%, humidity of 100%) for 24 hours while the culture dishes were gently shaken. The culture solution in each culture dish was then removed, and the culture dishes were gently washed with 10% FBS cell culture solution, and then viable cells adhered to the inner wall of the culture dishes were observed under a microscope (inverted fluorescence/visible microscope with cooled CCD camera, IX83, available from Olympus Corporation) by the fluorescent staining method of F-actin using viable cell staining by a fluorescent dye (CytoPainter F-actin Staining Kit, available from Abcam). The fluorescent staining method of F-action can be performed, for example, according to the method described in Vera D M, Robert J E, Ved P S, Orrin S and John S C, “Optimizing leading edge F-actin labeling using multiple actin probes, fixation methods and imaging modalities” BIOTECHNIQUES, VOL. 66, NO. 3, (2019), or in Michael M, Matthias P and Robert G “Actin visualization at a glance” n J. Cell Sci. 130, 525-530. (2017) doi:10.1242/jcs.20448.

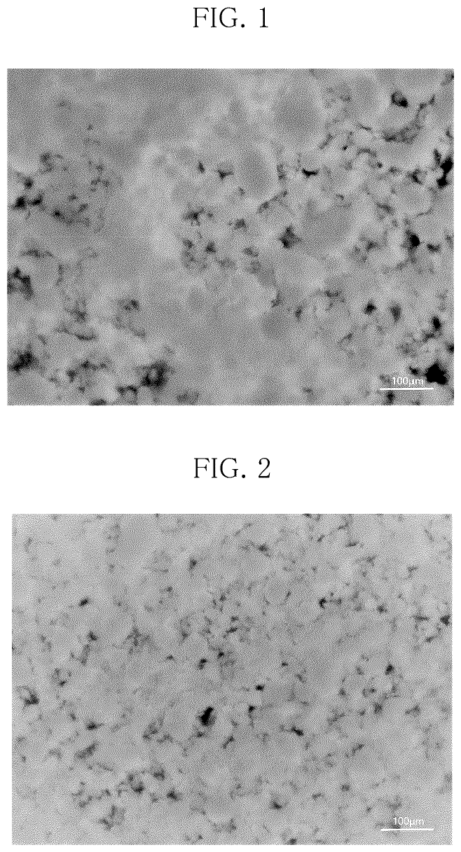

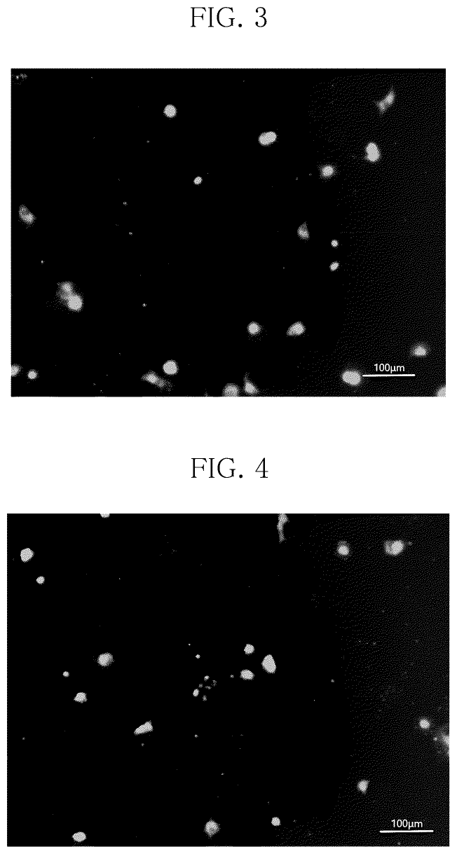

Microscopic images are shown in FIGS. 1 to 4. FIG. 1 shows a captured image of the surface of a potassium phosphate film when cells having been detached and suspended with DS are cultured on a culture dish with the potassium phosphate film. FIG. 2 shows a captured image of the surface of a potassium phosphate film when cells having been detached and suspended with heparin Na are cultured on a culture dish with the potassium phosphate film. FIG. 3 shows a captured image of the bottom surface of a culture dish when cells having been detached and suspended with DS are cultured on a culture dish without a potassium phosphate film. FIG. 4 shows a captured image of the bottom surface of a culture dish when cells having been detached and suspended with heparin Na are cultured on a culture dish without a potassium phosphate film. In FIGS. 1 to 4, the part that looks white or gray is a viable cell group stained with the fluorescent dye.

As shown in FIGS. 1 and 2, numerous viable cells were observed adhering to the surface of the calcium phosphate film, either alone or forming cell clusters. On the other hand, as shown in FIGS. 3 and 4, a very small number of viable cells adhered to the bottom surface of the culture dish without a calcium phosphate film. The same experiment was performed using Chinese hamster fibroblasts, and equivalent results were obtained. This demonstrates that calcium phosphate, which is a solid preparation, allows cells having been detached and suspended by the active ingredient such as DS or heparin Na to re-adhere and activates the cells. That is, by using a combination of a cell treatment agent containing an active ingredient and a predetermined preparation of multivalent cations, dormant cells having become dormant by the cell treatment agent can be awakened. Therefore, a set reagent combining these two is suitable for awakening dormant cells.

### Experimental Example 6: Relationship Between Viscosity and Concentration of Alginic Acid, and Cell Protective Effect

Using three types of alginic acid with different viscosities as an active ingredient in a cell treatment agent and a Ringer's solution as a conditioning solution for use, for example, in preservation and transportation of cells, the cell protective effects of sodium alginate with different viscosities were examined below. As the three types of alginic acid with different viscosities, (a) 1% aqueous solution of sodium alginate with a viscosity at 20° C. of 120 mPa s (high viscosity) (Snow Algin L, available from Fuji Chemical Industries Co., Ltd.), (b) 1% aqueous solution of sodium alginate with a viscosity at 20° C. of 30 mPa·s (low viscosity) (Snow Algin SSL, available from Fuji Chemical Industries Co., Ltd.), and (c) 10% aqueous solution of sodium alginate with a viscosity at 20° C. of 30 mPa·s (very low viscosity (extremely low viscosity)) (low molecular weight sodium alginate, available from Kyosei Pharmaceutical Co., Ltd.) were used. The Ringer's solution was the same as that used in Experimental Example 1.

(6-1) Preservation at Normal Temperature for 24 Hours

Cell preservation solutions were prepared by adding alginic acid of different viscosities described above to the Ringer's solution to give the concentrations shown in Table 15. To each cell preservation solution, the above-described rat corneal epithelial cells were added as cells at a concentration of 10⁶ cells/ml, and the resultant solution was left to stand in a room at normal temperature (22° C.) for 24 hours, and then whether cells were alive or dead was determined. Rates of viable cells (average survival rate (%)) were calculated and compared. Cell viability was determined by the trypan blue excretion test which is the ordinary method of confirming cell viability. The results are shown in Table 15. The results are of N=4.

TABLE 15

| | | | | Average survival |
|---|---|---|---|---|
| | | | | rate after |
| | | Sodium alginate | | preservation |
| | | Viscosity | | for 24 |
| | Conditioning | (measurement | Concentration | hours |
| Cell | solution | conditions) | (mg/ml) | (%) |
| Rat corneal | Ringer's | — | 0 | 36 |
| epithelial cell | solution | 120 mPa · s | 0.1 | 62 |
| | | (1% aqueous | | |
| | | solution, 20° C.) | 2.5 | 59 |
| | | 30 mPa · s | 0.2 | 50 |
| | | (1% aqueous | | |
| | | solution, 20° C.) | 1 | 54 |
| | | 30 mPa · s | 20 | 46 |
| | | (10% aqueous | 50 | 50 |
| | | solution, 20° C.) | | |

(6-2) Preservation Under Refrigeration for 168 Hours (7 Days)

The average survival rate (%) was determined in the same manner as in Experimental Example (6-1) except that the preservation conditions were changed to leaving still for 7 days in a refrigerator at a set temperature of 4° C. and concentrations of sodium alginate were set as shown in Table 16. The results are shown in Table 16. The results are of N=4.

TABLE 16

| | | | | Average survival |
|---|---|---|---|---|
| | | | | rate after |
| | | Sodium alginate | | preservation |
| | | Viscosity | | for 7 |
| | Conditioning | (measurement | Concentration | days |
| Cell | solution | conditions) | (mg/ml) | (%) |
| Rat corneal | Ringer's | — | 0 | 44 |
| epithelial cell | solution | 120 mPa · s | 0.5 | 53 |
| | | (1% aqueous | | |
| | | solution, 20° C.) | 2.5 | 52 |
| | | 30 mPa · s | 0.5 | 51 |
| | | (1% aqueous | | |
| | | solution, 20° C.) | 2.5 | 56 |
| | | 30 mPa · s | 2.5 | 54 |
| | | (10% aqueous | 10 | 69 |
| | | solution, 20° C.) | 50 | 72 |

As shown in Table 15, when the preservation time is relatively short (24 hours at room temperature of 22° C.), alginic acid with relatively high viscosity (1% aqueous solution, 120 mPa·s (20° C.)) (high viscosity) exerts the cell protective effect more effectively, compared to the cases of other viscosities (low viscosity, very low viscosity) in cell preservation solutions prepared at relatively low concentrations (herein, generally 2.5 mg/ml or less). As shown in Table 16, when cells are preserved for a very long time (7 days), alginic acid with very low viscosity (10% aqueous solution, 30 mPa·s (20° C.)) (very low viscosity) can exert the cell protective effect more effectively, compared to the cases of other viscosities (high viscosity, low viscosity) in cell preservation solutions prepared at relatively high concentrations (herein, generally 10 mg/ml or more). Table 16 also shows that alginic acid with very small viscosity is expected to improve the effect at still higher concentrations.

1. A cell treatment agent comprising at least one selected from the group consisting of alginic acid, heparins, dextran sulfate, and a proteolytic enzyme inhibitor as an active ingredient.

2. The cell treatment agent according to claim 1, comprising at least one selected from the group consisting of a cell culture solution, an extracellular fluid replacement solution, and a maintenance infusion.

3. The cell treatment agent according to claim 1, for cell dormancy of making a cell in tissue or a cultured cell dormant.

4. The cell treatment agent according to claim 1, for cell protection of maintaining viability of a cell in tissue or a cultured cell to prevent death of the cell.

5. The cell treatment agent according to claim 1, for cell preservation for maintaining a cell in an undifferentiated state or a low differentiated state.

6. The cell treatment agent according to claim 1, for cell detachment and suspension of detaching and suspending a cultured cell from a scaffold of a living tissue or from a scaffold of a culture substrate, or detaching and suspending a cell in tissue of a living tissue from a scaffold of the living tissue.

7. The cell treatment agent according to claim 1, the cell treatment agent being a liquid for cell preservation, tissue preservation, or organ preservation.

8. A set reagent for awakening of a dormant cell, the set reagent being a combination reagent of the cell treatment agent according to claim 1 and a solid or liquid preparation containing a polyvalent cation.

9. The cell treatment agent according to claim 1, comprising, as the active ingredient, the alginic acid and at least one selected from the group consisting of the heparins, the dextran sulfate, and the proteolytic enzyme inhibitor.

10. The cell treatment agent according to claim 1, comprising, as the active ingredient, the heparins and at least one selected from the group consisting of the dextran sulfate, and the proteolytic enzyme inhibitor.

11. The cell treatment agent according to claim 1, comprising the dextran sulfate and the proteolytic enzyme inhibitor, as the active ingredient.

12. The cell treatment agent according to claim 3, comprising at least one selected from the group consisting of the alginic acid, the heparins, and the proteolytic enzyme inhibitor, as the active ingredient.

13. The cell treatment agent according to claim 5, comprising the alginic acid, as the active ingredient.

14. The cell treatment agent according to claim 6, comprising, as the active ingredient, at least one selected from the group consisting of the alginic acid, the heparins, and the proteolytic enzyme inhibitor.

15. A method of making a cell in tissue or a cultured cell dormant, the method using a cell treatment agent comprising, as an active ingredient, at least one selected from the group consisting of alginic acid, heparins, and a proteolytic enzyme inhibitor.

16. A method of detaching and suspending a cultured cell from a scaffold of a living tissue or from a scaffold of a culture substrate, or of detaching and suspending a cell in tissue of a living tissue from a scaffold of the living tissue, the method using a cell treatment agent comprising, as an active ingredient, at least one selected from the group consisting of alginic acid, heparins, and a proteolytic enzyme inhibitor.

17. A method for awakening a dormant cell, by activating a solid or liquid preparation containing a polyvalent cation after making the cell dormant using the cell treatment agent according to claim 1.
